(19) United States
(12) Patent Application Publication
(10) Pub. No.: US 2023/0032568 A1
(43) Pub. Date: Feb. 2, 2023

(54) ELECTRONIC DEVICE, METHOD AND COMPUTER PROGRAM FOR OLFACTORY ASSESSMENT OF A PRODUCT STATE

(71) Applicant: ARYBALLE, GRENOBLE (FR)

(72) Inventor: Kirill ARKHIPOV, GRENOBLE (FR)

(21) Appl. No.: 17/791,768

(22) PCT Filed: Jan. 8, 2021
(86) PCT No.: PCT/FR2021/050028

§ 371 Date: Jul. 8, 2022

(30) Foreign Application Priority Data
Jan. 10, 2020 (FR) FR2000226

(51) Int. Cl. G01N 21/552; G01N 21/27; G01N 33/00

(52) U.S. Cl. CPC G01N 21/554; G01N 21/274; G01N 33/0062; G01N 2033/0068

(57) ABSTRACT

An electronic device for assessing a state of a product likely to transform by emission of volatile organic compounds includes olfactory sensors, designed to provide signals representative of a presence of volatile organic compounds in the ambient air close to the product, and a processor for processing the provided signals to obtain a signature representative of the state of the product. It further includes a memory for storing a reference signature, representative of an exposure of the olfactory sensors to a reference humid environment in which the product is not present, and a computer which computes a similarity value for similarity between the signature representative of the state of the product and the reference signature, to provide a product transformation index value from the computed similarity value.

The present invention relates to an electronic device for assessing a state of a product likely to transform by emission of volatile organic compounds. It also relates to a method implemented by such a device and a corresponding computer program.

The invention applies more particularly to an electronic device for assessing a state of a product likely to transform by emission of volatile organic compounds, comprising:
several olfactory sensors designed to:
   interact respectively with several volatile organic compounds likely to be present in the ambient air when these olfactory sensors are placed close to the product,
   provide signals representative of a presence of these volatile organic compounds in the ambient air; and
a processor for processing the signals provided by the olfactory sensors in order to obtain N component(s) of a signature representative of the state of the product.

This kind of device can be envisaged for various applications of olfactory assessment of the state of a product likely to transform by emission of volatile organic compounds. Generally speaking, the transformation of the product must be understood as being due to a microbiological or enzymatic activity. The possible applications can also extend to the aerobic or anaerobic transformation of living organisms. The state of the product should be understood as being a state of transformation of the product, i.e., freshness, or conversely degradation, maturation, fermentation, etc., depending on the applications.

Such a device is, for example, marketed by the company Aryballe Technologies under the name NeOse Pro (registered trademark) since 2018. It is more generally an odor identification device, generally called an “electronic nose”, whose processor for processing the signals provided by the olfactory sensors is capable of producing a digital signature, or recognition fingerprint, specific to each detected odor. Its operation is for example described in the patent documents FR 3 063 543 A1 and FR 3 071 061 B1. In general, the number N is equal to the number of olfactory sensors.

By training on different products and under different conditions, this device is potentially able to identify all the odors, and in particular those that are characteristic of the freshness of a product such as an edible piece of dead animal flesh in a situation of degradation.

Without further analysis, this learning process, which depends on the input parameters of an artificial intelligence system, can be tedious and complex, and even a source of inaccuracies and errors.

It may thus be desirable to provide a device for assessing a state of a product that makes it possible to avoid at least some of the above-mentioned problems and constraints.

It is therefore proposed an electronic device for assessing a state of a product likely to transform by emission of volatile organic compounds, comprising:
several olfactory sensors designed to:
   interact respectively with several volatile organic compounds likely to be present in the ambient air when these olfactory sensors are placed close to the product,
   provide signals representative of a presence of these volatile organic compounds in the ambient air; and
a processor for processing the signals provided by the olfactory sensors in order to obtain N≥1 component(s) of a signature representative of the state of the product;
   further comprising:
a memory for storing a reference signature with N component(s) representative of an exposure of the olfactory sensors to a reference humid environment in which the product is not present;
a computer which computes a similarity value for similarity between the N component(s) of the signature representative of the state of the product and that(those) of the reference signature, to provide a product transformation index value from the computed similarity value.

Thus, the invention takes advantage of an unexpected and surprising finding that the state of a product likely to transform by emission of volatile organic compounds is in fact determinable with the aid of the electronic device defined above by simple comparison with an exposure of its olfactory sensors to a reference humid environment. The similarity between the two signatures compared is indeed characteristic of the state of transformation of the product. The closer they are, the closer the product is to a reference state in which it does not emit particular volatile organic compounds, especially the fresher it is. The further away they are, the more the product is in a state of advanced transformation, especially advanced degradation. As a result of this finding, the learning that may be implemented to improve the operation of the device is greatly simplified. It has also been observed that the resulting estimates are more reliable. It should be noted that, according to the applications envisaged, the transformation index can be understood as an index of freshness, or conversely of degradation, maturation, fermentation, etc.

Also proposed is a method for assessing a state of a product likely to transform by emission of volatile organic compounds, comprising the following steps:
exposure of the olfactory sensors of an electronic device according to the invention to a reference humid environment in which the product is not present;
processing of the signals provided by the olfactory sensors when they are exposed to the reference humid environment to obtain N component(s) of a reference signature;
exposure of the olfactory sensors to the ambient air when they are placed close to the product;
processing of the signals provided by the olfactory sensors when they are placed close to the product to obtain N component(s) of a signature representative of the state of the product; and
computing of a similarity value for similarity between the N component(s) of the signature representative of the state of the product and that(those) of the reference signature, for providing a product transformation index value from the computed similarity value.

Optionally, the exposure of the olfactory sensors to the ambient air when they are placed close to the product comprises successively:
a referential phase of exposure of the olfactory sensors to a dry air environment without product;
an analytical phase of exposure of the olfactory sensors to the volatile organic compounds emitted by the product;
a final phase, called desorption, of re-exposure of the olfactory sensors to the dry air environment without product;
   and the processing of the provided signals comprises, for each of N signal(s) obtained from the signals provided by the olfactory sensors of the device, the computing of a statistical value, representative of the considered signal in a predetermined time window, as a component of the signature representative of the product state.

Also optionally, the processing of the signals provided by the olfactory sensors when they are placed close to the product comprises taking into account each of the N signal(s) obtained within a predetermined time window which is at the end of the analytical phase.

Also optionally, the processing of the signals provided by the olfactory sensors when they are placed close to the product comprises taking into account each of the N signal(s) obtained within a predetermined time window which is at the beginning of the desorption phase.

Also optionally, a method for assessing the freshness of a product according to the invention may comprise a selection step for selecting, from among the olfactory sensors of the electronic device, a subset of sensors sensitive to volatile nitrogenous, nitro-nitrogenous and/or sulfurous components.

Also optionally, the similarity value is a distance value, e.g., an N-Euclidean distance, between signatures.

Also optionally, a method for assessing a state of a product according to the invention may comprise a calibration step including a learning process carried out on several products of different degrees of transformation and known in advance in order to associate their respectively computed similarity values with predetermined values of transformation index.

Also optionally, the signal processing comprises obtaining N signal(s) representative of the interactions between the volatile organic compounds emitted by the product and the olfactory sensors of the electronic device, this obtaining being from one of the devices of the set consisting of:
a plasmon resonance amplification device;
a Mach-Zehnder interferometric amplification device; and
an amplification device using functionalized resonant membranes.

It is also proposed a computer program downloadable from a communication network and/or stored on a medium readable by a computer and/or executable by a processor, comprising instructions for executing the processing and computing steps for a method for assessing a state of a product according to the invention, when said program is executed on a computer.

The invention will be better understood with the aid of the following description, given solely by way of example and made with reference to the appended drawings in which:

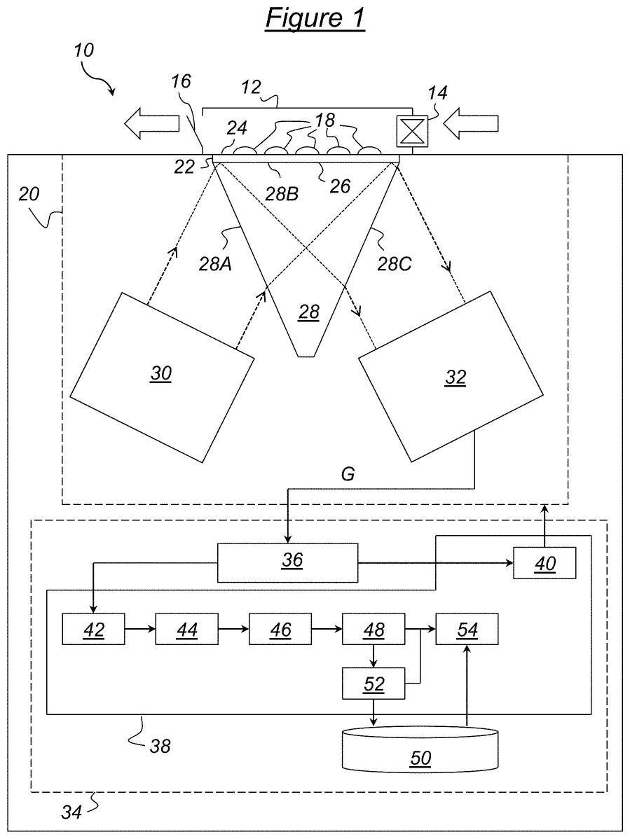

FIG. 1 diagrammatically represents the general structure of an electronic device for assessing a state of a product, according to an embodiment of the invention,

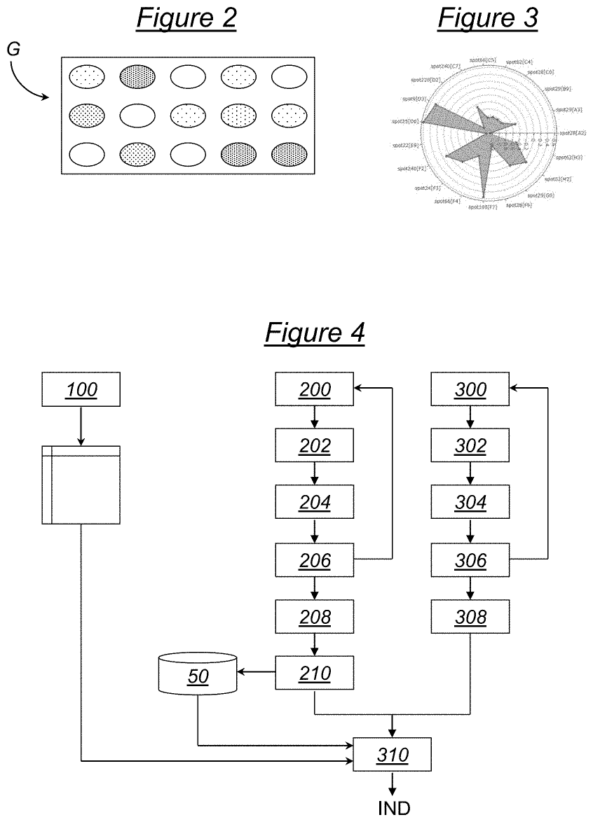

FIG. 2 illustrates a typical example of an image produced by the electronic device of FIG. 1, in which the olfactory sensors of this device are visible,

FIG. 3 illustrates, in the form of a pie chart, an example of an olfactory signature that can be calculated by the device of FIG. 1,

FIG. 4 illustrates the successive steps of a method for assessing a state of a product, according to an embodiment of the invention,

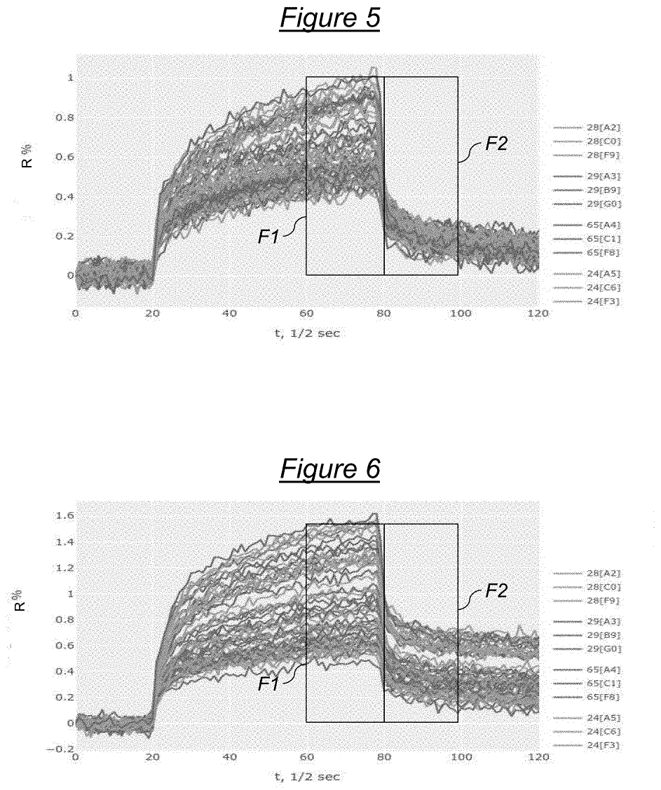

FIG. 5 illustrates, in the form of superimposed time diagrams, an example of reference reflectance signals that can be obtained by the device of FIG. 1, and

FIG. 6 illustrates, in the form of superimposed time diagrams, an example of reflectance signals that can be obtained by the device of FIG. 1 for a product emitting olfactory components that are characteristic of a given transformation.

The electronic device 10 for odor identification, and more specifically for assessing a freshness state of a product, diagrammatically represented in FIG. 1 comprises a chamber 12 for receiving ambient air. To do this, it comprises a suction device 14 designed to suck in air from outside the chamber 12 and bring it inside. It further comprises an air outlet 16 which can be selectively closed to keep the ambient air in the chamber 12 or opened to allow the ambient air to be evacuated from the chamber 12 and renewed by the activation of the suction device 14.

In its chamber 12, the device 10 comprises several olfactory sensors 18, for example about sixty, designed to interact with volatile organic compounds likely to be present in the ambient air of the chamber 102 when they are placed in the vicinity of a product which is in a situation of transformation, for example degradation, by emission of these compounds, in particular when the suction device 14 is in the vicinity of the considered product. Each olfactory sensor 18 is, for example, a biosensor designed to interact with compounds of a particular family of compounds. In practice, each olfactory sensor 18 may comprise a molecule, such as a peptide, complementary to the compounds of the family associated with that olfactory sensor 18.

Further associated with a surface plasmon resonance imaging system 20, i.e., an SPR (Surface Plasmonic Resonance) amplification system, the olfactory sensors 18 are designed to provide signals representative of a presence, in the ambient air of the chamber 12, of the volatile organic compounds with which they may interact.

More specifically, the imaging system 20 comprises a metallic layer 22, such as gold, comprising a first side 24 facing into the chamber 12 so as to contact the ambient air therein. The olfactory sensors 18 are fixed to this first side 24 at predefined positions. In the example described, the sensors are arranged in a matrix on a positioning grid, i.e., they are respectively located at the centers of cells of this grid. The metal layer 22 further has a second side 26 opposite the first side 24.

The imaging system 20 further comprises a prism 28 comprising a light input side 28A, a side 28B against which the second side 26 of the metal layer 22 extends and a light output side 28C.

The imaging system 20 further comprises an illumination device 30 designed to illuminate the second side 26 of the metal layer 22 with collimated and polarized light. Specifically, the collimated and polarized light is emitted from the illumination device 30 through the light input side 28A of the prism 28 to the second side 26 of the metal layer 22.

Since the second side 26 of the metal layer 22 has a certain reflectance, i.e., a property of reflecting a fraction of the light it receives, a portion of the collimated and polarized light is reflected. However, the illumination device 30 is further designed to produce a surface plasmon resonance on the first side 24 of the metal layer 22. This resonance decreases the reflectance of the second side 26 and is sensitive to the refractive index of the ambient air present up to about 100 nanometers above the first side 24, and thus in particular above the olfactory sensors 18 which have a smaller thickness. However, the interaction of a compound with any of the sensors 18 modifies the refractive index of the air above this sensor and thus decreases the reflectance of the second side 26 of the metal layer 22 under this sensor.

Thus, the reflectance of the second side 26 of the metal layer 22 varies locally under each olfactory sensor 18 as a function of the compound(s) interacting with that sensor.

To produce plasmon resonance, the illumination device 30 is preferably designed to emit transverse magnetic polarization light, denoted TM, i.e., having a magnetic field parallel to the second side 26 of the metal layer 22. The illumination device 30 may further be designed to emit transverse electric polarized light, denoted TE, i.e., having an electric field parallel to the second side 26 of the metal layer 22, on command instead of TM light. In addition, the prism 28 serves to obtain an angle of incidence on arrival at the metal layer 22 (i.e., when the prism 28 is present, at the interface between the prism glass 28 and the metal layer 22) allowing the resonance of surface plasmons.

The imaging system 108 further comprises a camera 32 arranged to receive light emitted from the illumination device 30, after reflection from the second side 26 of the metal layer 22 and passage through the light output side 28C of the prism 28. The camera 32 is designed to provide a sequence of images G of the sensors 18 from the received light. Each sequence of images represents the set of signals provided by the olfactory sensors. In the example described, each image is a luminance image with pixel values expressed as scalar values, so that each image is in grayscale.

FIG. 2 illustrates a typical diagrammatic example of an image G produced by the camera 32 of the electronic device of FIG. 1, in which the olfactory sensors 18 of this device are visible with their luminance values indicative of the volatile organic compounds with which they may have interacted. It will be noted that in the example described, the sensors 18 are circular. But because of the inclination of the camera 32 with respect to the second surface 26 of the metal layer 22, the areas they occupy in the image G are ellipses.

It should be noted that the non-limiting example of FIG. 1 illustrates the obtaining of signals representative of the interactions between the volatile organic compounds emitted by the product and the olfactory sensors 18 of the electronic device 10 using an optical index variation amplification device of the SPR type, but this could be replaced by an optical index variation amplification device by Mach-Zehnder interferometry, a mechanical resonance frequency variation amplification device by functionalized resonant membranes (for example in MEMS or NEMS technology), or any other equivalent physical transduction device (i.e. optical, mechanical, etc.), by making a simple adaptation of the electronic device 10 which will not be described because it is within the reach of the skilled person. The general idea remains to functionalize the olfactory sensors 18 (i.e., biosensors, polymers, carbon nanotubes, etc.) in such a way that they differentially adsorb and desorb volatile organic compounds, to form a differentiated molecular interaction response of the olfactory sensors, and to amplify the response using a physical transduction device.

With reference to FIG. 1 again, the device 10 further comprises several functional modules that will be described below. In the example described, these modules are software. Thus, the device 10 comprises a computer-like element 34 comprising a processing unit 36 and an associated memory area 38 in which a plurality of computer programs or a plurality of functions of a single computer program are stored. These computer programs comprise instructions designed to be executed by the processing unit 36 to perform the functions of the software modules. They are presented as distinct, but this distinction is purely functional. They could just as easily be grouped in any combination into one or more software programs. Their functions could also be at least in part micro-programmed or micro-wired into dedicated integrated circuits, such as digital circuits. Thus, in variant, the computer 34 could be replaced by an electronic device composed only of digital circuits (without computer program) for the realization of the same actions.

The device 10 thus comprises firstly a software module 40, to be executed by the processing unit 36, for controlling the suction device 14, the air outlet 16 and the imaging system 20.

It further comprises optionally but advantageously a software module 42, to be executed by the processing unit 36, for selecting, among the olfactory sensors 18 of the electronic device 10, a subset of sensors sensitive to volatile components characteristic of the freshness of the considered product. These characteristic volatile components may vary from one product to another, so that the selection of olfactory sensors made by the software module 42 may also vary and be parameterized. Quite generally, when the product is a piece of animal flesh, it is advantageous to select olfactory sensors that are sensitive to nitrogenous, nitro-nitrogenous and/or sulfurous volatile components. The selected subset comprises for example N 1 olfactory sensor(s), in particular advantageously several olfactory sensors (N 2).

The device 10 further comprises a software module 44, to be executed by the processing unit 36, for extracting N reflectance signals respectively representative of the interactions of the N selected olfactory sensors with the relevant volatile organic compounds from the luminance values specific to these N selected olfactory sensors in an image sequence G provided by the camera 32. These reflectance signals are, for example, expressed as a percentage according to a ratio of luminance values obtained with transversely polarized light to luminance values obtained with the same light polarized at 90 degrees for each of the N selected olfactory sensors.

The device 10 further comprises a software module 46, to be executed by the processing unit 36, for selecting a time window for analyzing the N reflectance signals with a view to extracting N components of an olfactory signature representative of a state of freshness of the studied product.

The device 10 further comprises a software module 48, to be executed by the processing unit 36, for obtaining the N components of the aforementioned olfactory signature from the N reflectance signals. This obtaining may include a correction of the N reflectance signals extracted in the selected time window. This correction will be detailed later with reference to FIG. 3. It comprises mainly two components: a correction of drift of the olfactory sensors which is well known and will not be detailed, as well as a correction by subtraction of a reference frame which depends on a specific methodology of exposure of the olfactory sensors to the ambient air when they are placed close to the studied product. It allows to obtain N corrected reflectance signals. Each of the N components of the signature results for example directly or indirectly from the calculation of a statistical value representative of a respective one of the N corrected reflectance signals in the selected time window. It can simply be a scalar average value in this time window. It can also be a more complex statistical value, scalar or vector.

An example of a 19-component olfactory signature represented in a circular diagram is shown in FIG. 3.

In accordance with the general principles of the present invention, the device 10 comprises a memory area 50 for storing a reference signature with N components representative of an exposure of the olfactory sensors 18 to a reference humid environment without the presence of the product under study. This reference signature is obtained by sequentially executing the software modules 42 to 48 at least once. Its N components result from the same selection and processing as defined above for exposure of the olfactory sensors 18 to the product under study. By humid environment, we mean an ambient air comprising a significant mass fraction of water vapor, i.e., greater than 3000 ppm (part per million), or even greater than 4000 ppm, and advantageously greater than 4500 ppm, which is equivalent to a relative humidity greater than 90% at 4° C. The temperature of 4° C. is taken as a reference because it is a good, but non-limiting, example of the temperature of a refrigerated environment in which a product intended for consumption is usually kept and whose freshness is monitored.

The sequential execution can in particular be repeated several times to obtain several successive reference signatures that can then be statistically processed, in particular by an average calculation or the like, to obtain an improved reference signature intended to be stored in memory 50. To this end, the device 10 optionally but advantageously comprises a software module 52, to be executed by the processing unit 36, for statistical processing of several signatures with N components. In particular, it can be a simple averager of the N reference signature components to obtain a reference centroid signature.

The device 10 further comprises a software module 54, to be executed by the processing unit 36, for computing a similarity value between the N components of the signature representative of the freshness status of the product and those of the reference signature stored in memory 50. The similarity value is, for example, a distance value, among other possibilities an N-Euclidean distance, between signatures. This software module 54 is further designed to provide a product transformation index value, including a product freshness index from the computed similarity value. It can be the similarity value itself, or a calibrated value resulting from a learning process carried out on several products of different degrees of freshness and known in advance to associate their respectively calculated similarity values with predetermined values of the freshness index.

The use of the electronic device 10 for the implementation of a method for assessing a state of a product likely to transform by emission of volatile organic compounds will now be detailed with reference to FIG. 4.

A prior and optional calibration step 100 consists in carrying out a learning process on several products of different degrees of transformation, in particular of different degrees of freshness, known in advance, in order to associate their similarity values, respectively calculated by execution of the software modules 42 to 54, with freshness index values which are a priori assigned to them and which are chosen to have a meaning for a user. A smart correspondence between computable similarity values and freshness index values that are more easily understood by the user is thus created. It is in this way that the freshness index resulting from the execution of the software modules 42 to 54 on a product whose freshness is not known can be considered as calibrated by using this learning. It should be noted of course that this smart correspondence can be declined according to different classes of products and different conditions of temperature, pressure, humidity or others.

In a step 200, the electronic device 10 is arranged so that its olfactory sensors 18 can be exposed to a reference humid environment without the presence of any product that may transform by emission of volatile organic compounds. More specifically, this step may comprise a first referential phase of exposure of the olfactory sensors 18 to a dry air environment without product, then a second analytical phase of exposure of the olfactory sensors 18 to the reference humid environment, then a third final phase, known as desorption, of re-exposure of the olfactory sensors 18 to the dry air environment without product. During these three exposure phases, the camera 32 produces a sequence of images G which it transmits to the computer 34. By dry environment is meant an ambient air comprising a low mass fraction of water vapor, i.e., less than 500 ppm (part per million), or even less than 100 ppm, and advantageously less than 10 ppm, which is equivalent to a relative humidity of less than 0.1% at 4° C. Such dry air can be obtained for example by using silica gel or by extracting air in a frozen environment.

In a subsequent step 202, the image sequence G is received by the computer 34. The processing unit 36 then executes software modules 42 and 44 to obtain N reflectance signals representative of the image sequence G for each of the N selected olfactory sensors. This results in, for example, N temporal signals such as those shown in FIG. 5, where the three exposure phases defined above are clearly visible: the first referential phase extending from 0 to about 20 on the x-axis (expressed in half-seconds, i.e., for example, according to a 2 Hz time sampling), the second analytical phase extending from about 20 to about 140, and the third desorption phase beginning at about 140.

In a subsequent step 204, the processing unit 36 executes the software module 46 for selecting a time window for analyzing the reflectance signals such as those in FIG. 5. In particular, it was noted that two time windows were particularly relevant for this type of three-phase exposure of the olfactory sensors 18. A first relevant time window F1 is relative to the end of the analytical phase and extends, for example, over samples 120 to 140 in the example signals of FIG. 5. A second relevant time window F2 relates to the beginning of the desorption phase and extends for example over samples 140 to 170 in the example signals of FIG. 5. One of these two time windows is therefore advantageously selected at this stage. Alternatively, several time windows could be selected at this stage to obtain a more complex signature with vector components, in particular the two windows F1 and F2.

In a subsequent step 206, the processing unit 36 executes the software module 48 to obtain a signature with N components. In particular, given the aforementioned three-phase exposure of the olfactory sensors 18, the aforementioned reference frame subtraction correction may involve subtracting the observed offset of each of the reflectance signals in the referential phase from the respective values of those signals in the analytical phase. For each reflectance signal, this offset is, for example, the average of the signal values in the referential phase.

Steps 200-206 can be repeated as many times as desired, without amending the selections chosen in steps 202 and 204, to obtain multiple N-component signatures.

In a subsequent step 208, if steps 200-206 have been performed multiple times, the processing unit 36 executes the software module 52 for statistical processing of the resulting signatures and obtaining a reference signature, for example averaged, which is then stored in memory 50 in a step 210.

In a step 300, the electronic device 10 is arranged so that its olfactory sensors 18 are exposed to the ambient air when they are placed close to a product that is likely to transform, for example be degraded, by emission of volatile organic compounds. More precisely, this step comprises the same referential, analytical and desorption phases as those of step 200, except that the analytical phase is a phase of exposure of the olfactory sensors 18 to the volatile organic compounds emitted by the product under study. During these three exposure phases, the camera 32 produces a new sequence of images G which it transmits to the computer 34.

In a subsequent step 302, the image sequence G is received by the computer 34. The processing unit 36 then executes the software modules 42 and 44, with the same selection of N olfactory sensors, to obtain N reflectance signals representative of the image sequence G for each of the N selected olfactory sensors. Thus, for example, N temporal signals such as those shown in FIG. 6 are obtained. We can see very clearly, as in FIG. 5, the three exposure phases mentioned above. It can also be noticed that the windows F1 and F2 are particularly discriminating to distinguish a product in advanced state of degradation from the reference situation of FIG. 5. In particular, the reflectance signals are distributed in a clearly different way in the window F1 of end of analytical phase between FIGS. 5 and 6, whereas the return to the situation of the referential phase in desorption phase is clearly slower in FIG. 6 than in FIG. 5, with moreover a more important residual signal.

In a subsequent step 304, the processing unit 36 executes the software module 46 to select the same analysis time window as in step 204.

In a subsequent step 306, the processing unit 36 executes the software module 48 to obtain N components of a signature representative of a state of transformation, in particular of freshness of the studied product.

Steps 300 to 306 can be repeated as many times as desired, without changing the selections chosen in steps 302 and 304, to obtain several signatures representative of the freshness state of the product under study.

In a subsequent step 308, if steps 300-306 have been performed multiple times, the processing unit 36 executes the software module 52 for statistical processing of the resulting signatures and obtaining a final representative signature, for example averaged.

Finally, during a last step 310, the processing unit 36 executes the software module 54 to calculate a similarity value between the N components of the signature representative of the state of freshness of the studied product and those of the reference signature stored in memory 50 and to deduce a transformation index value “IND”, in particular of freshness of the product, from the calculated similarity value. This deduction can be carried out by exploiting the learning of the prior step 100 if necessary.

It clearly appears that an electronic device for assessing a state of a product such as the one described above makes it possible to obtain relevant and precise estimates for a particularly simple operation with limited learning.

It should also be noted that the invention is not limited to the embodiment described above.

In particular, a multi-component olfactory signature implementation has been described, but a very simple single-component implementation (N=1) could be imagined, in particular by selecting a single olfactory sensor. However, it seems advantageous to improve the performances of the device by increasing the number of components in each signature, in particular by increasing the number of olfactory sensors used for the freshness assessment.

The electronic device 10 shown in FIG. 1 is furthermore depicted as a single, compact device integrating the sensors 18 of the chamber 12, the imaging system 20, the functional modules with processing units and computer programs, and the signature storage memory 50. However, these elements are separable in multiple variants. In particular, the functional modules and the memory 50 can be easily moved remotely, knowing that they must remain capable of receiving the signals from the sensors via the imaging system 20 or any other equivalent transduction system.

It will be more generally apparent to those skilled in the art that various modifications can be made thereto, in the light of the teaching just disclosed. In the above detailed presentation of the invention, the terms used should not be interpreted as limiting the invention to the embodiments set forth in the present description, but should be interpreted to include all equivalents the anticipation of which is within the reach of those skilled in the art by applying their general knowledge to the implementation of the teaching just disclosed to them.

1. An electronic device for assessing a state of a product likely to transform by emission of volatile organic compounds, comprising:
several olfactory sensors configured to:
interact respectively with several volatile organic compounds likely to be present in the ambient air when these olfactory sensors are placed close to the product,
provide signals representative of a presence of these volatile organic compounds in the ambient air;
a processor for processing the signals provided by the olfactory sensors in order to obtain N≥1 component(s) of a signature representative of the state of the product;
a memory for storing a reference signature with N component(s) representative of an exposure of the olfactory sensors to a reference humid environment in which the product is not present, and
a computer which is adapted to compute a similarity value for similarity between the N component(s) of the signature representative of the state of the product and that(those) of the reference signature, to provide a product transformation index value from the computed similarity value.

2. A method for assessing a state of a product likely to transform by emission of volatile organic compounds, comprising:
exposure of the olfactory sensors of an electronic device according to claim 1 to a reference humid environment in which the product is not present;
processing of the signals provided by the olfactory sensors when the olfactory sensors are exposed to the reference humid environment to obtain N component(s) of a reference signature;
exposure of the olfactory sensors to the ambient air when they the olfactory sensors are placed close to the product;
processing of the signals provided by the olfactory sensors when the olfactory sensors are placed close to the product to obtain N component(s) of a signature representative of the state of the product; and
computing of a similarity value for similarity between the N component(s) of the signature representative of the state of the product and that(those) of the reference signature, for providing a product transformation index value from the computed similarity value.

3. The method for assessing a state of a product according to claim 2, wherein the exposure of the olfactory sensors to the ambient air when placed close to the product comprises successively:
a referential phase of exposure of the olfactory sensors to a dry air environment without product;
an analytical phase of exposure of the olfactory sensors to the volatile organic compounds emitted by the product;
a final phase, called desorption, of re-exposure of the olfactory sensors to the dry air environment without product;
and wherein the processing of the provided signals comprises, for each of N signal(s) obtained from the signals provided by the olfactory sensors of the device, the computing of a statistical value, representative of the considered signal in a predetermined time window, as a component of the signature representative of the product state.

4. The method for assessing a state of a product according to claim 3, wherein the processing of the signals provided by the olfactory sensors when the olfactory sensors are placed close to the product comprises taking into account each of the N signal(s) obtained within a predetermined time window which is at the end of the analytical phase.

5. The method for assessing a state of a product according to claim 3, wherein the processing of the signals provided by the olfactory sensors when the olfactory sensors are placed close to the product comprises taking into account each of the N signal(s) obtained within a predetermined time window which is at the beginning of the desorption phase.

6. The method for assessing a state of a product according to claim 2, comprising selecting, from among the olfactory sensors of the electronic device, a subset of sensors sensitive to volatile nitrogenous, nitro-nitrogenous and/or sulfurous components.

7. The method for assessing a state of a product according to claim 2, wherein the similarity value is a distance value between signatures.

8. The method for assessing a state of a product according to claim 2, comprising a calibration step including a learning process carried out on several products of different degrees of transformation and known in advance in order to associate their respectively computed similarity values with predetermined values of transformation index.

9. The method for assessing a state of a product according to claim 2, wherein the signal processing comprises obtaining N signal(s) representative of the interactions between the volatile organic compounds emitted by the product and the olfactory sensors of the electronic device, said obtaining being from one of the devices of the set consisting of:
a plasmon resonance amplification device;
a Mach-Zehnder interferometric amplification device; and
an amplification device using functionalized resonant membranes.

10. A non-transitory computer readable medium readable by a computer and/or executable by a processor, comprising instructions for executing the processing and computing steps of a method for assessing a state of a product according to claim 2, when said instructions are executed on a computer.

11. The method for assessing a state of a product according to claim 7, wherein the distance value is an N-Euclidean distance.
